(19) United States
(12) Patent Application Publication
(10) Pub. No.: US 2004/0102477 A1
(43) Pub. Date: May 27, 2004

(54) Polymorphic forms of (S)-Repaglinide and the processes for preparation thereof

(75) Inventors: Manne Satyanarayana Reddy, Hyderabad (IN); Sajja Eswaraiah, Hyderabad (IN); Mathad Vijayavitthal Trippapnchar, Hyderabad (IN); Govindan Shanmugam, Hyderabad (IN); Maddipatla Madhavi, Hyderabad (IN); Kolla Naveen Kumar, Hyderabad (IN)

Correspondence Address: Janet I. Cord, Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/647,449

(22) Filed: Aug. 25, 2003

(30) Foreign Application Priority Data
Aug. 23, 2002 (IN) 621/MAS/2002
Aug. 30, 2002 (IN) 637/MAS/2002

(51) Int. Cl. A61K 31/445; C07D 211/34

(52) U.S. Cl. 514/317; 546/238

(57) ABSTRACT

The present invention relates to crystalline and amorphous forms of repaglinide and the process for the preparation thereof. In particular, a new crystalline Form III of S-repaglinide is described and the process for its preparation is provided. Both the crystalline and amorphous forms of repaglinide are suitable for pharmaceutical purposes in the treatment of diabetes. The processes of the invention are simple, non-hazardous and commercially suitable.

### CROSS-REFERENCE TO RELATED APPLICATION

This application claims priority of Indian Patent Application No. 621/MAS/2002, filed Aug. 23, 2002, and Indian Patent Application No. 637/MAS/2002, filed Aug. 30, 2002, the disclosures of which are incorporated herein by reference in their entirety.

### FIELD OF THE INVENTION

The present invention relates to improved processes for the preparation of both crystalline Form I and Form-II of (S)-2-Ethoxy-4-[N-(1-(2-piperidino-phenyl)-3-methyl-1-butyl)-amino carbonyl methyl] benzoic acid [(S)-Repaglinide].

The present invention also relates to a new crystalline Form III of (S)-repaglinide, the process for preparation of the crystalline Form-III of (S)-repaglinide, and compositions containing crystalline Form-III of (S)-repaglinide.

The present invention also relates to amorphous form of S-repaglinide, the process for preparation of the amorphous form of S-repaglinide, and compositions containing the amorphous form of S-repaglinide.

### BACKGROUND OF THE INVENTION

The drug repaglinide (S)-2-Ethoxy-4-[N-(1-(2-piperidino-phenyl)-3-methyl-1-butyl)-amino carbonyl methyl] benzoic acid [(S)-Repaglinide] is used in treatment of diabetes. It belongs to the meglitinide class of insulin secretagogues, compounds which stimulate insulin release from the pancreas. Meglitinides tend to be rapid onset compounds with short duration of action, making them particularly suitable for administration just before meals. In general, preparation of repaglinide and certain of its polymorphic forms are known in the art. However, it is also known that different polymorphic forms of the same drug may have substantial differences in certain pharmaceutically important properties. Therefore, there is a continuing need for new solid forms of repaglinide and new methods of preparation.

### SUMMARY OF THE INVENTION

In accordance with one aspect, the invention provides a new crystalline Form III of S-repaglinide. Preferably, the crystalline Form III of S-repaglinide has an X-ray diffraction pattern, expressed in terms of 2 theta angles, that includes five or more peaks selected from the group consisting of 7.80±0.09, 19.25±0.09, 13.46±0.09, 21.19±0.09, 4.44±0.09, 12.92±0.09, 20.0±0.09, 19.59±0.09, 20.34±0.09, 18.06±0.09, 22.18±0.09, 15.71±0.09, 17.08±0.09, 9.28±0.09, 14.34±0.09, 18.75±0.09, 23.77±0.09, 25.32±0.09, 22.58±0.09, 11.09±0.09, 11.89±0.09, 24.08±0.09, 25.02±0.09, 30.26±0.09, 23.24±0.09, 28.03±0.09, 16.24±0.09, 25.78±0.09, 6.81±0.09, 26.68±0.09, 27.34±0.09, 35.50±0.09, 38.74±0.09 degrees. More preferably, crystalline Form III of repaglinide has substantially the same X-ray diffraction pattern as shown in FIG. 1. Various embodiments and variants are provided.

In accordance with yet another aspect, the invention provides a composition that contains repaglinide in a solid form, wherein at least 80% by weight of the solid repaglinide is its crystalline Form III having an X-ray diffraction pattern, expressed in terms of 2 theta angles, that includes five or more peaks selected from the group consisting of 7.80±0.09, 19.25±0.09, 13.46±0.09, 21.19±0.09, 4.44±0.09, 12.92±0.09, 20.0±0.09, 19.59±0.09, 20.34±0.09, 18.06±0.09, 22.18±0.09, 15.71±0.09, 17.08±0.09, 9.28±0.09, 14.34±0.09, 18.75±0.09, 23.77±0.09, 25.32±0.09, 22.58±0.09, 11.09±0.09, 11.89±0.09, 24.08±0.09, 25.02±0.09, 30.26±0.09, 23.24±0.09, 28.03±0.09, 16.24±0.09, 25.78±0.09, 6.81±0.09, 26.68±0.09, 27.34±0.09, 35.50±0.09, 38.74±0.09 degrees. Various embodiments and variants are provided.

In accordance with yet another aspect, the invention provides a process for making the crystalline Form III of (S) repaglinide, the process including a) providing a solution of repaglinide in a haloalkane solvent; b) contacting the solution with C₅-C₁₀ aliphatic or alicyclic hydrocarbon to form a precipitate; and c) isolating the precipitate, which is the crystalline Form III of (S) repaglinide. Various embodiments and variants are provided. The process is believed to be simple, eco-friendly and cost-effective.

In accordance with another aspect, the invention provides a process for preparation of crystalline Form II of (S)-repaglinide that includes a) providing a solution of S-repaglinide in a solvent containing aromatic hydrocarbon with the proviso that the solvent does not include petroleum ether; b) cooling the solution to separate a solid mass; and c) isolating the solid mass which is the crystalline Form II of S-repaglinide. Methods for preparation of Form I of S-repaglinide are also provided. Various embodiments and variants are provided.

In accordance with yet another aspect, the invention provides a pharmaceutical composition that includes crystalline Form III of (S)-repaglinide and a pharmaceutically acceptable carrier or diluent. Preferably, the pharmaceutical composition is a solid dosage form for oral administration.

In accordance with yet another aspect, the present invention also relates to the amorphous form of (S)-Repaglinide, the process for preparation of the amorphous form of (S)-Repaglinide, and compositions containing amorphous form of (S)-Repaglinide.

In accordance with another aspect, the invention provides a pharmaceutical composition that includes a prophylactically or therapeutically effective amount of the amorphous form of (S)-repaglinide that is substantially free of its crystalline form and one or more pharmaceutically acceptable excipients. The pharmaceutical compositions of this aspect of the invention may be formulated, for example, as solid dosage forms for oral administration. In accordance with yet another aspect, the invention provides a composition containing a solid form of repaglinide, which is at least 80% amorphous. In accordance with yet another aspect, the invention provides a process for preparation of an amorphous form of (S)-repaglinide. In one embodiment of this aspect of the invention, the process involves dissolution of (S) repaglinide in providing a solution of repaglinide in an alcohol solvent; cooling the solvent until a precipitate is formed; and isolating the precipitate which is the amorphous. Pharmaceutical compositions that include a prophylactically or therapeutically effective amount of the amorphous form of (S) repaglinide produced by the process described, and one or more pharmaceutically acceptable excipients are also provided.

### BRIEF DESCRIPTION OF THE ACCOMPANYING DRAWINGS

FIG. 1 shows a sample X-ray powder diffractogram of the crystalline Form-Ill (S)-repaglinide.

FIG. 2 is a characteristic differential scanning colorimetry thermogram of the crystalline Form III of (S)-repaglinide.

FIG. 3 is a characteristic infrared spectrum of the crystalline Form III of (S)-repaglinide.

FIG. 4 shows a sample X-ray powder diffractogram of amorphous form of (S)-repaglinide.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

The crystalline compound designated herein as “crystalline Form III”, and referred to hereinafter as a crystalline Form III of (S)-Repaglinide, is a new crystalline polymorph of repaglinide different from known polymorphs. It is characterized via X-ray powder diffraction, DSC and/or infrared spectroscopy. It is further described below.

“Pharmaceutically acceptable” means that which is useful in preparing a pharmaceutical composition that is generally non-toxic and is not biologically undesirable and includes that which is acceptable for veterinary use and/or human pharmaceutical use.

“Anti-solvent” is a solvent which when added to an existing solution of a substance reduces the solubility of the substance.

The term “composition” includes, but is not limited to, a powder, a solution, a suspension, a gel, an ointment, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art.

Repaglinide has the chemical structure

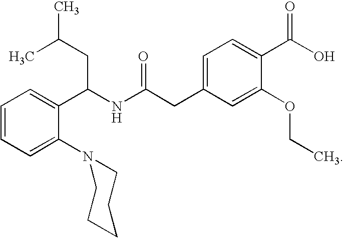

U.S. Pat. No. 5,216,167, incorporated herein by reference in its entirety, claims repaglinide, its enantiomers, and their pharmaceutically acceptable salts. The '167 patent also discloses three polymorphic forms for racemic repaglinide, which were designated as Form-A, Form-B, and Form-C. Form A (melting range of 90-92° C.) was obtained via recrystallization from acetone/petroleum ether. Form B (melting range of 140-142° C.) was obtained via recrystallization from ethanol/water. Form C melting range of (74-85° C.) was re-precipitated from methanol.

U.S. Pat. No. 5,312,924 described the preparation of S-enantiomer of repaglinide via resolution of racemic 3-methyl-1-(2-piperidinophenyl)-1-butyl amine with N-acetyl-L-glutamic acid to afford the (S)-enantiomer of corresponding amine. The resultant amine was then reacted with 3-ethoxy-4-ethoxy carbonylphenyl acetic acid to give ethyl ester of (S)-repaglinide. The ethyl ester of (S)-repaglinide on saponification (S)-repaglinide. The '924 patent, incorporated herein by reference in its entirety and for the purpose of describing the preparation, discloses the preparation of (S)-repaglinide. One polymorph of S-repaglinide was obtained via re-crystallization from ethanol/water (melting point 130-131° C.). Another polymorph of S-repaglinide was obtained by recrystallization from petroleum ether/toluene (melting point 99-101° C.).

Different solid forms of the same drug may exhibit different properties, including characteristics that have functional implications with respect to their use as active ingredients of pharmaceutical products. For example, polymorphs of the same drug may have substantial differences in such pharmaceutically important properties as dissolution rates and bioavailability. Likewise, different polymorphs may have different processing properties, such as hydroscopicity, flowability, and the like, which could affect their suitability as active pharmaceuticals for commercial production.

The inventors have prepared and analyzed the polymorphs described in the prior art by X-ray diffraction spectroscopy. Thus, the inventors re-precipitated S-repaglinide using the solvent systems described in the '167 patent for the racemic material and characterized the resulting solids. The crystalline forms obtained by recrystallizing S-repaglinide from acetone/pet ether and ethanol/water are found to be same as indicated by their X-ray diffraction patterns (Table 1):

TABLE 1

| | |
|---|---|
| Acetone/Petroleum ether | Ethanol/water |

| 2-Theta | Intensity % | 2-Theta | Intensity % |
|---|---|---|---|
| 7.58 | 100 | 7.60 | 100 |
| 10.06 | 61.1 | 10.08 | 67.6 |
| 12.40 | 2.0 | 12.40 | 1.9 |
| 12.98 | 9.5 | 13.02 | 9.4 |
| 13.21 | 15.9 | 13.22 | 17.3 |
| 13.75 | 25.2 | 13.76 | 27.5 |
| 14.56 | 7.5 | 14.58 | 7.5 |
| 15.26 | 7.0 | 15.28 | 6.7 |
| 15.53 | 1.1 | 15.53 | 1.0 |
| 6.65 | 31.7 | 16.66 | 33.7 |
| 6.94 | 3.7 | 16.94 | 3.9 |
| 7.51 | 8.4 | 17.53 | 8.8 |
| 8.56 | 12.4 | 18.58 | 12.5 |
| 0.26 | 58.5 | 20.27 | 62.4 |
| 0.48 | 19.3 | 20.52 | 21.6 |
| 1.37 | 0.8 | 21.41 | 0.9 |
| 1.88 | 1.2 | 21.89 | 1.3 |
| 2.94 | 25.4 | 22.93 | 24.8 |
| 3.35 | 5.3 | 23.35 | 7.4 |
| 3.95 | 19.7 | 23.96 | 20.2 |
| 5.02 | 1.6 | 24.99 | 1.1 |
| 5.36 | 2.4 | 25.33 | 2.2 |
| 5.66 | 4.2 | 25.67 | 4.1 |
| 6.23 | 3.5 | 26.25 | 3.8 |
| 6.65 | 8.4 | 26.65 | 9.3 |
| 7.75 | 7.9 | 27.76 | 8.4 |
| 8.73 | 0.9 | 28.73 | 1.1 |
| 9.47 | 1.1 | 29.46 | 1.7 |
| 9.77 | 2.8 | 29.77 | 2.9 |
| 0.86 | 15.3 | 30.88 | 16.7 |
| 1.61 | 1.3 | 31.56 | 1.0 |
| 2.49 | 0.8 | 32.49 | 0.9 |
| 5.46 | 1.4 | 35.46 | 1.1 |
| 6.09 | 0.9 | 36.07 | 1.1 |
| 7.02 | 1.8 | 37.04 | 1.9 |
| 8.84 | 1.8 | 38.89 | 2.4 |
| 9.48 | 1.1 | 39.49 | 1.0 |
| 3.55 | 1.1 | 43.52 | 1.0 |
| 4.08 | 1.1 | 44.09 | 0.9 |

This prior art polymorph of S-repaglinide (melting point 130-131° C.) is designated herein as crystalline Form I.

The inventors also re-precipitated another polymorph of S-repaglinide as described in the prior art (toluene/petroleum ether) and characterized the resulting solid by XRD (Table 2).

TABLE 2
Crystalline Form II obtained as per the process
disclosed is USP '167

| 2-Theta | Intensity % |
|---|---|
| 7.586 | 100 |
| 5.415 | 83.3 |
| 19.566 | 44.2 |
| 16.352 | 38.2 |
| 22.939 | 29.5 |
| 25.938 | 15.3 |
| 21.044 | 14.5 |
| 10.564 | 13.1 |
| 14.039 | 12 |
| 18.812 | 10.9 |
| 11.025 | 9 |
| 27.558 | 5.3 |
| 23.698 | 4.6 |
| 12.102 | 4.3 |
| 20.267 | 4.2 |
| 9.104 | 3.7 |
| 17.05 | 2.8 |
| 15.324 | 2.5 |
| 12.701 | 2.3 |
| 9.696 | 1.5 |
| 29.052 | 1.5 |
| 30.107 | 1.4 |
| 31.22 | 1.1 |
| 33.113 | 1.1 |

This prior art polymorph of S-repaglinide (melting point 99-101° C.) is designated herein as crystalline Form II.

In one aspect, the invention provides an improved process for the preparation of the crystalline Form-II of (S)-repaglinide. In the process of this aspect of the invention, first, a solution of S-repaglinide is provided in a solvent that contains aromatic hydrocarbon but does not include petroleum ether in contrast to the process of the prior art. While it is contemplated that solvent components other than the aromatic hydrocarbon may be used, preferably such additional components do not include any aliphatic hydrocarbon components. More preferably, the solvent consists only of the aromatic hydrocarbon. Examples of aromatic hydrocarbons include benzene, naphthalene, anthracene, furan, thiophene, pyrroles, oxazoles, thiazoles, triazoles, imidazoles, pyridazine, pyridine, purines, pyrimidine, triazine, thiazine, indoles, quinolines, indenes, azulene, porphines, and any of the above rings which are fused with other rings or substituted. Preferred aromatic hydrocarbons are benzene and substituted benzenes, the substituted benzenes preferably substituted with an alkyl group. More preferred aromatic hydrocarbons include, but are not limited to, benzene, toluene, ethyl benzene and xylene. Mixtures of aromatic hydrocarbons may be used. In a specific preferred variant, a single aromatic hydrocarbon is used. The more preferred single aromatic hydrocarbon is toluene.

The preferred method for providing the solution of S-repaglinide involves mixing a powder of the starting S-repaglinide with the solvent and heating the mixture until the solution is formed. The powder of the starting S-repaglinide may be selected from any crystalline and amorphous form of S-repaglinide. Of course, crude S-repaglinide, in either powder, oil, or any other form, may also be used to form the solution. To form the solution, the mixture is heated to 50-100° C., preferably to 70-75° C. At this point, the solution may be filtered to remove any un-dissolved and/or extraneous matter. Then, the solution is cooled with stirring to until a solid mass separates therefrom. The solid mass, which is the crystalline Form II of S-repaglinide, is isolated, typically by filtration, and dried to provide a dry powder of the crystalline Form II of S-repaglinide.

In a specific embodiment of this aspect, the process involves crystallizing S-repaglinide from toluene without petroleum ether utilized in the prior art by

a) dissolving crystalline or an amorphous form of (S)-Repaglinide in aromatic hydrocarbon solvents comprising of benzene, toluene, ethyl benzene or xylene, preferably toluene at a temperature of 50-100° C., preferably at 70-75° C.;

b) cooling the reaction solution;

c) stirring the solution till the solid substantially separates;

d) filtering the separated solid by conventional methods;

e) drying the resulting solid under vacuum at a temperature of 30 to 90° C. to a constant weight to afford the crystalline Form-II of (S)—Repaglinide.

The inventors obtained a X-ray diffraction pattern of the solid powder of crystalline Form II of S-repuglinide produced via the process of this aspect of the invention (Table 3).

TABLE 3

| 2-Theta | Intensity % |
|---|---|
| 7.57 | 100 |
| 5.383 | 60.6 |
| 19.527 | 40.3 |
| 16.311 | 25 |
| 22.904 | 21.4 |
| 20.979 | 11.1 |
| 25.871 | 10.5 |
| 10.551 | 10 |
| 14.007 | 10 |
| 18.775 | 8.6 |
| 10.966 | 6.8 |
| 12.066 | 3.7 |
| 27.524 | 3.7 |
| 23.65 | 3.2 |
| 9.081 | 2.8 |
| 20.18 | 2.5 |
| 15.222 | 2.3 |
| 17.174 | 2 |
| 14.461 | 1.6 |
| 12.689 | 1.4 |
| 34.979 | 1.3 |
| 29.08 | 1 |
| 31.165 | 1 |
| 33.074 | 1 |
| 9.648 | 0.9 |
| 30.088 | 0.8 |
| 31.934 | 0.4 |

The X-ray diffractogam was measured on a Bruker Axs, D8 Advance Powder X-ray Diffractometer with Cu K alpha-I Radiation source. The powder of (S)-Repaglinide was also characterized by Differential Scanning Colorimetry thermogram, which was analyzed on Schimadzu differential scanning calorimeter in a temperature range of 25-160° C. with a heating rate of 5° C./minute under Nitrogen with a flow rate of 50.0 ml/minute. The Differential Scanning Colorimetry thermogram of (S)-Repaglinide exhibits a significant endo pattern with peaks around 105° C. and 132° C.

As can be seen from a comparison between Tables 2 and 3, the solid obtained by the process described herein is the crystalline Form II of S-repaglinide. However, the process of this aspect of the present invention provides certain distinct advantages over the prior art. In particular, for the single-solvent embodiment of this aspect of the invention, the process of the invention allows easy recovery of the solvent via distillation, with the possibility of subsequent re-use. This permits more economically efficient industrial manufacturing of the crystalline Form II of S-repaglinide, with a minimum solvent loss and smaller effluent streams. The yield of the crystalline Form II of S-repaglinide is also much higher than the prior art process. The crystalline Form-II obtained has high purity (e.g. >99.0%) and the powder obtained via the process is free-flowing and non-solvated crystalline solid and hence may be useful in the preparation of pharmaceutical formulations.

In another aspect, the present invention relates to an improved process for the preparation of Form-I of Repaglinide. The process comprises the dissolution of the crude Repaglinide in a solvent chosen from the list comprising C₁-C₅ alcoholic solvents such as ethanol, methanol, prepanol, terbutanol, n-butanol, isopropanol or ketone solvents such as acetone, diethyl ketone, methyl isobutyl ketone, methyl ethyl ketone or ether solvents such as tetrahydrofuran or other solvents like ethyl acetate, acetonitrile at ambient temperature, followed by addition of suitable anti solvent chosen from a list comprising ether solvents, such as methyl tert-butyl ether, isopropyl ether or hydrocarbons solvents, such as o-xylene, n-heptane, n-hexane or petroleum ether or water.

The crystalline nature of the crystalline Form-I of Repaglinide obtained by the present processes is characterized by the powder X-ray powder diffraction, which confirmed its nature as the Form I. The X-ray diffractogram of crystalline Form-I of Repaglinide of the present invention is measured on Bruker Axs, D8 Advance powder X-ray Diffractometer with Cu K alpha-1 Radiation source (Table 4).

TABLE 4

| 2-Theta | Intensity |
|---|---|
| Value (°) | I/IO (%) |
| 7.598 | 100 |
| 20.28 | 74.3 |
| 10.066 | 71.1 |
| 20.447 | 59.2 |
| 22.884 | 50.8 |
| 18.557 | 40.9 |
| 12.97 | 40.1 |
| 13.748 | 38.1 |
| 16.644 | 32.8 |
| 14.559 | 32.7 |
| 13.218 | 26.1 |
| 23.925 | 22.8 |
| 16.915 | 16.1 |
| 30.866 | 14.2 |
| 22.515 | 12.1 |
| 12.375 | 12 |
| 17.508 | 10.2 |
| 26.654 | 9.4 |
| 25.654 | 8.8 |
| 26.19 | 8.5 |
| 27.732 | 8 |
| 15.25 | 7.4 |
| 24.982 | 6.4 |
| 29.736 | 4.5 |
| 21.431 | 4.3 |
| 15.522 | 3.9 |
| 11.171 | 3 |
| 21.879 | 3 |
| 35.496 | 2 |
| 36.177 | 1.6 |

Differential Scanning Colorimetry thermogram and Infrared spectra of the crystalline Form-I of S-repaglinide produced by the process of this aspect of the invention were also obtained. The Differential Scanning Colorimetry thermogram exhibits a significant endo peak around 124° C. Significant IR bands are appeared around 539.1, 617.9, 648.5, 697.1, 760.8, 781.8, 862.0, 920.0, 940.0. 983.3, 1040.0, 1090.7, 1112.2, 1148.9, 1216.4, 1299.8, 1340.2, 1383.0, 1448.4, 1490.4, 1567.5, 1607.2 1637.2, 1688.6, 1718.4, 1773.4, 1793.6, 1846.1, 2344.1, 2371.3, 2804.6, 2865.9, 2934.4 and 3.307.4 cm⁻¹.

In a particular variant of this aspect of the invention, the process for preparation of crystalline Form I of S-repaglinide is provided and includes:

a. dissolving the crude or crystalline or amorphous S-repaglinide in a solvent chosen from the list comprising C₁-C₅ chained or branched alcoholic solvents such as ethanol, methanol, propanol, ter-butanol, n-butanol, isopropanol or lower ketone solvents such as acetone, diethyl ketone, methyl isobutyl ketone, methyl ethyl ketone or halogenated hydrocarbon solvents such as chloroform or other solvents comprising tetrahydrofuran, ethyl acetate or acetonitrile at a temperature of 40-50° C.;

b. stirring the solution till clear solution results;

c. adding suitable anti-solvent chosen from a list comprising of C₂-C₇ branched or chained ether solvents such as methyl tert-butyl ether, isopropyl ether or C₃-C₇ branched or chained or aromatic hydrocarbon solvents, such as o-xylene, n-heptane, n-hexane, pet. ether or water;

d. optionally stirring the reaction mass obtained in step (c) for 0.5 to 10 hrs, more preferably for 1-2 hours at 30-50° C.;

e. cooling the contents of step (d) to 0-35° C., preferably 0-5° C. accompanied by gentle stirring for 0.5-10 hours, preferably for 1-2 hours, to afford a solid mass;

f. filtering the solid mass by conventional techniques, accompanied by washing with a solvent of step (a); and

g. drying the isolated compound, of step (f) at 30-100° C., preferably at 40-50° C. to a constant weight to afford the crystalline Form-I of S-repaglinide.

The starting material, crude or crystalline amorphous S-repaglinide can be prepared as per procedures known in prior art. List of the solvents and anti solvents, which can use in preparation of crystalline Form-I of S-repaglinide are depicted in Table 5.

TABLE 5

| Solvent | Anti solvent |
|---|---|
| Ethanol | Water |
| Acetone | Pet. ether |
| Methanol | Water |
| THF | MTBE |
| EtoAc | Pet. ether |
| n-propanol | Water |
| ACN | Water |
| MIBK | MTBE |
| Diethyl ketone | MTBE |
| Ter-Butanol | Water |
| Methyl ethyl ketone | n-heptane |
| Diglyme | n-heptane |
| Methyl | MTBE |
| ethyl ketone | |
| 1,4-dioxane | n-heptane |
| n-butanol | MTBE |
| CHCI₃ | n-hexane |

A new crystalline form of repaglinide has also been discovered. While the invention is not limited to any specific theory or preparation methodology, the inventors found that crystallization of repaglinide from a haloalkane solvent with a suitable anti-solvent produces a polymorph that is different from known polymorphs of Form I and II. The new polymorph is herein designated as the crystalline Form III of repaglinide. The preparation of the crystalline Form III is described in greater details below. The new crystalline Form III may be identified and differentiated by X-ray diffraction, differential scanning calorimetry (DSC) and/or infrared spectroscopy.

The crystalline Form III of S-repaglinide may be characterized by X-ray powder diffraction. The ray diffraction patterns are unique for the particular crystalline form. Each crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(α1) wavelength using the Bragg equation well known to those of skill in the art.

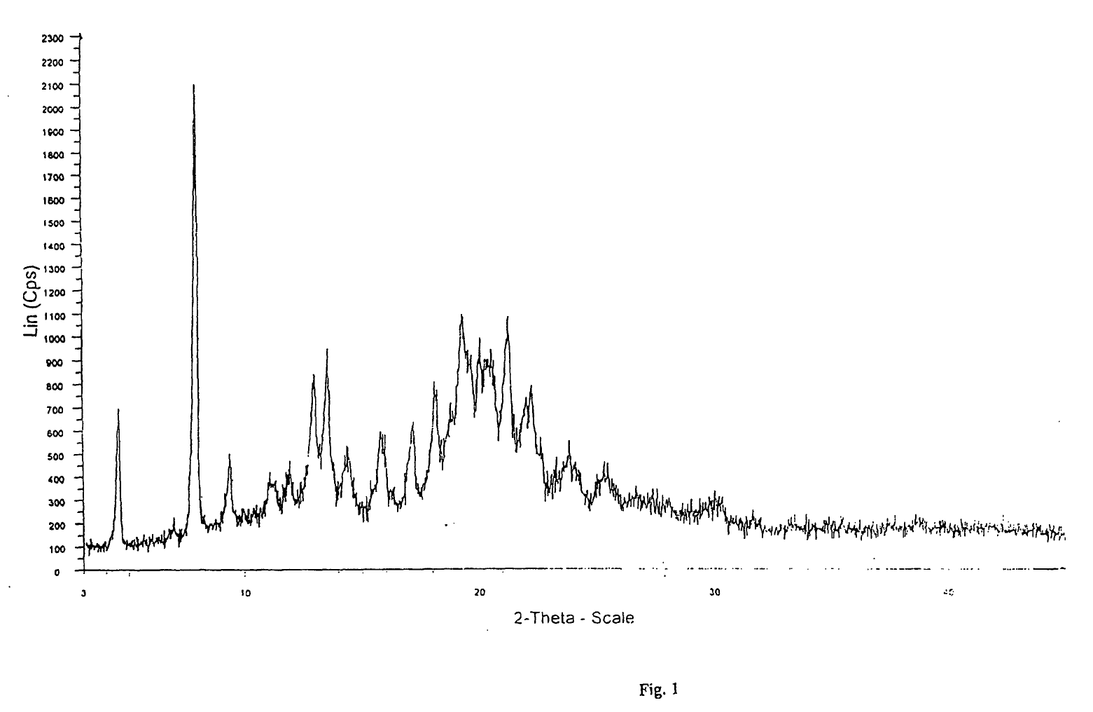

FIG. 1 shows an example of X-ray powder diffractogram of the crystalline Form-III (S)-Repaglinide obtained on a Bruker Axs, D8 Advance Powder X-ray Diffractometer with Cu K alpha-1 Radiation source. The pattern of X-ray diffraction peaks for crystalline Form-III of (S)-Repaglinide is shown in Table 6.

TABLE 6

| 2 Theta | Intensity (%) |
|---|---|
| 7.803 | 100.00 |
| 19.251 | 34.10 |
| 13.464 | 31.70 |
| 21.185 | 31.00 |
| 4.438 | 29.20 |
| 12.918 | 28.00 |
| 19.995 | 23.20 |
| 19.594 | 22.60 |
| 20.339 | 22.10 |
| 18.060 | 20.80 |
| 22.180 | 18.40 |
| 15.766 | 17.40 |
| 17.081 | 17.20 |
| 9.277 | 14.60 |
| 14.342 | 11.80 |
| 18.747 | 11.60 |
| 23.767 | 8.80 |
| 25.315 | 7.20 |
| 22.581 | 7.10 |
| 11.091 | 6.80 |
| 11.888 | 6.80 |
| 24.080 | 6.30 |
| 25.020 | 4.40 |
| 30.255 | 3.90 |
| 23.235 | 3.60 |
| 28.028 | 3.20 |
| 16.236 | 3.00 |
| 25.780 | 3.00 |
| 6.811 | 2.70 |
| 26.675 | 2.60 |
| 27.388 | 2.20 |
| 35.500 | 2.20 |
| 38.739 | 2.10 |

It should be kept in mind that slight variations in observed 2 theta angles or d-spacing values are expected based on the specific diffractometer employed, the analyst, and the sample preparation technique. More variation is expected for the relative peak intensities. Identification of the exact crystalline form of a compound should be based primarily on observed 2 theta angles with lesser importance attributed to relative peak intensities.

Some margin of error is present in each of the 2 theta angle assignments reported herein. The assigned margin of error in the 2 theta angles for Form III of repaglinide is approximately ±0.09 for each of the peak assignments. In view of the assigned margin of error, in a preferred variant, the crystalline Form III of repaglinide may be characterized by an X-ray powder diffraction patterns that includes five or more peaks selected from the group consisting of peaks with 2 theta angles of 4.44±0.09, 6.81±0.09, 7.80±0.09, 9.28±0.09, 11.09±0.09, 11.89±0.09, 12.92±0.09, 13.46±0.09, 14.34±0.09, 15.77±0.09, 16.24±0.09, 17.08±0.09, 18.06±0.09, 18.75±0.09, 19.25±0.09, 19.59±0.09, 19.99±0.09, 20.34±0.09, 21.18±0.09, 21.96±0.09, 22.18±0.09, 22.58±0.09, 23.24±0.09, 23.77±0.09, 24.08±0.09, 25.02±0.09, 25.31±0.09, 25.78±0.09, 26.67±0.09, 27.39±0.09, 28.03±0.09, 30.26±0.09, 35.50±0.09, and 38.74±0.09 degrees.

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of repaglinide obtained using the methods described herein, over FIG. 1 and readily determine whether the X-ray diffraction pattern of the unidentified form is substantially the same as the X-ray powder diffraction pattern of Form III. If the X-ray powder diffraction pattern is substantially the same as FIG. 1, the previously unknown crystalline form of repaglinide can be readily and accurately identified as Form III.

Although 2 theta angles or d-spacing values are the primary methods of identifying the crystalline form, it may be desirable to also compare relative peak intensities. As noted above, relative peak intensities may vary depending upon the specific diffractometer employed and the analyst's sample preparation technique. The peak intensities are reported as intensities relative to the peak intensity of the strongest peak.

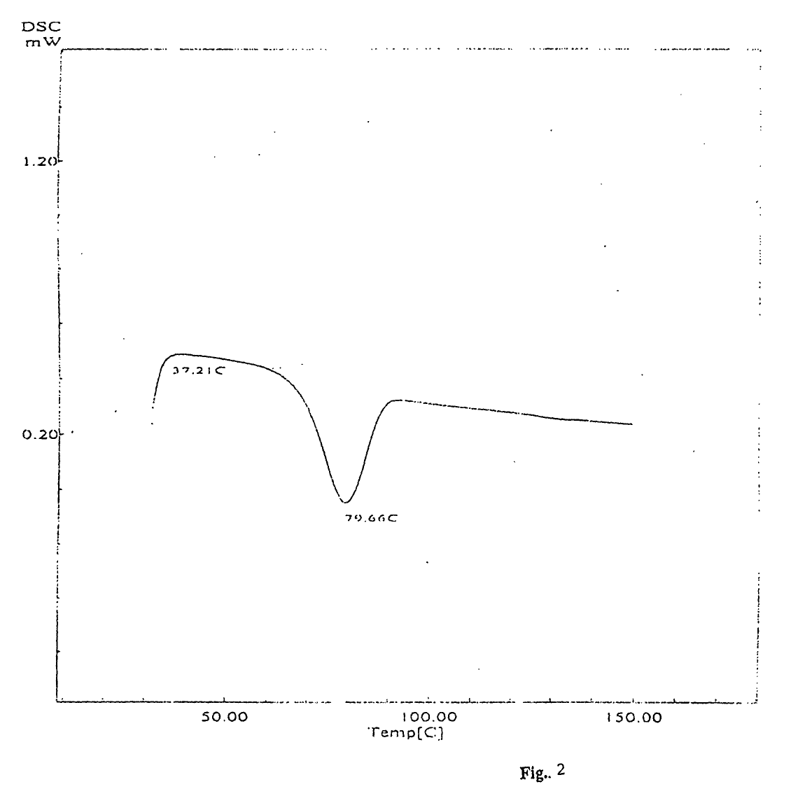

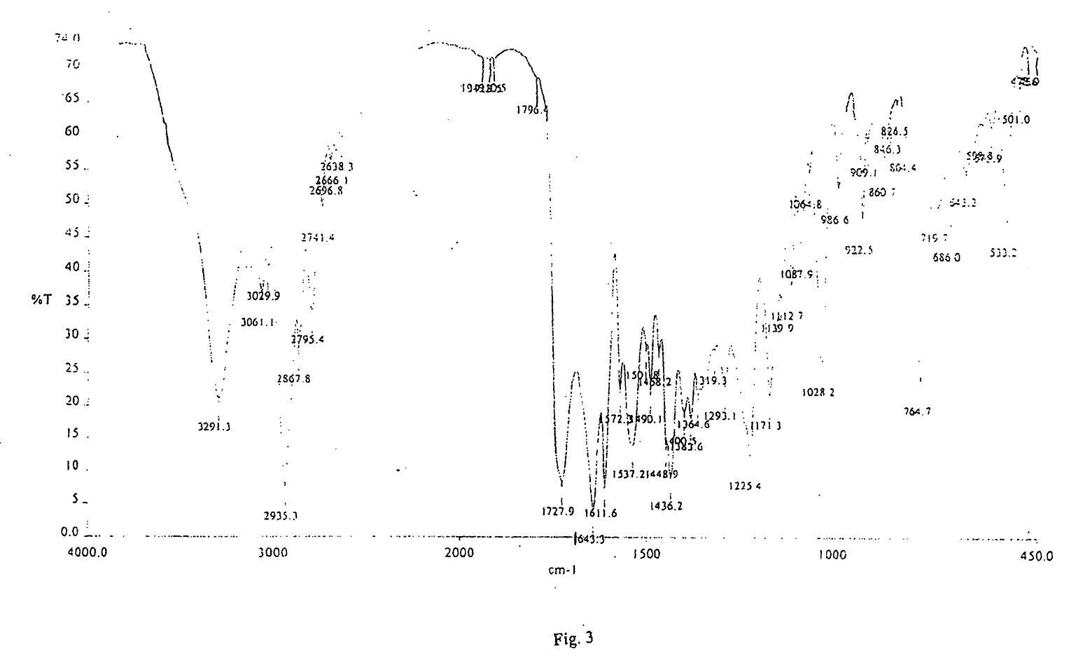

The crystalline form of repaglinide may be also characterized by differential scanning calorimetry and/or infrared spectroscopy. The DSC thermogram of crystalline Form III of repaglinide obtained by the inventors is shown in FIG. 2. It exhibits a significant endo pattern with identified peaks at about 80° C. The DSC thermogram of FIG. 2 was measured on Schimadzu diffrential scanning colorimeter in a temperature range of 50-250° C. with a heating rate of 5° C./minute. The infrared spectrum of crystalline Form III of repaglinide obtained by the inventors is shown in FIG. 3. It was measured on Perkin-Elmer FT-IR instrument by KBr-transmission method. The significant bands may be identified at about 3291 cm⁻¹, about 3029 cm⁻¹, about 2935 cm⁻¹, about 2795 cm⁻¹, about 1292 cm⁻¹, about 1727 cm⁻¹, about 1643 cm⁻¹, about 1611 cm⁻¹, about 1537 cm⁻¹, about 1436 cm⁻¹, about 1225 cm⁻¹, 1175 cm⁻¹, 1087 cm⁻¹, 1028 cm⁻¹, 986 cm⁻¹, 922 cm⁻¹, 860 cm⁻¹, 764 cm⁻¹, 686 cm⁻¹, and about 533 cm⁻¹.

The invention also provides a composition containing solid S-repaglinide, of which at least 80%, by total weight of the solid repaglinide in the composition, is its crystalline Form III. The preferred form of this composition is solid S-repaglinide powder suitable for use as active ingredient in formulating pharmaceutical products. The remainder of the solid S-repaglinide in the composition, i.e., 20% or less of the total weight of repaglinide may be, for example, the crystalline form of repaglinide. In one specific embodiment, the composition contains at least 90% of the crystalline Form III of S-repaglinide with respect to total weight of the solid repaglinide in the composition. In another specific embodiment, the composition contains at least 95% of the crystalline Form III of S-repaglinide with respect to total weight of the solid S-repaglinide in the composition. In yet another embodiment, the composition is substantially free of any forms of S-repaglinide other than its crystalline Form III. In yet another embodiment, in addition to crystalline Form III, the composition includes at least a small amount of crystalline Forms I or II of S-repaglinide, or both. In a non-limiting example, the composition includes 95% of crystalline Form III of S-repaglinide and at least 1% of other crystalline forms of repaglinide. In another non-limiting example, the composition includes at least 80% of crystalline Form III of S-repaglinide and at least 5% of crystalline Forms I and/or II of S-repaglinide. All compositions, in 0.1% increments, which include at least 80% of crystalline Form III of S-repaglinide and at least 1% of other crystalline forms of repaglinide, are contemplated. All percentages are based upon the total amount of the solid repaglinide in the composition.

X-ray diffraction provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of crystalline repaglinide can be determined in an unknown composition. Preferably, the measurements are made on solid powder repaglinide. The X-ray powder diffraction patterns of an unknown composition can be compared to known quantitative standards containing pure crystalline forms of repaglinide (e.g., Forms I or II) to identify the percent ratio of the crystalline form of repaglinide. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of crystalline repaglinide. The calibration curve may be created in a manner known to those of skill in the art. For example, five or more artificial mixtures of crystalline forms of repaglinide, at different amounts, may be prepared. In a non-limiting example, such mixtures may contain, 2%, 5%, 7%, 8%, and 10% of repaglinide for each crystalline form. Then, X-ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of crystalline repaglinide in an unknown sample. For the unknown mixture of crystalline and amorphous repaglinide, the intensities of the selected characteristic peak(s) in the mixture, relative to an intensity of this peak in a calibration mixture, may be used to determine the percentage of the given crystalline form in the composition, with the remainder determined to be the amorphous material.

A process for preparation of the crystalline Form III of S-repaglinide is also provided. The process of this aspect of the invention involves formation of a solution of S-repaglinide in a haloalkane solvent, followed by treating the solution with C₅-C₁₀ aliphatic or alicyclic hydrocarbon anti-solvent to form a precipitate, and isolating the precipitate, which is the crystalline Form III of S-repaglinide, from the suspension. The contacting step preferably includes adding the C₅-C₁₀ aliphatic or alicyclic hydrocarbon to the solution; but the order of addition may also be reversed. Preferred haloalkane solvents include dichloromethane, chloroform, and dichloroethane. C₅-C₇ aliphatic or alicyclic hydrocarbons are the preferred anti-solvent. Examples of suitable anti-solvents include, but are not limited to, petroleum ether, hexane, n-heptane, cyclohexane, and cycloheptane. Both the concentration of the starting S-repaglinide solution and the ration of solvent and anti-solvent may vary. In one variant, the concentration of the starting solution ranges from about 0.25 gram of S-repaglinide to about 1 gram per milliliter of the haloalkane solvent. In another variant, the concentration of the starting solution ranges from about 0.4 gram of S-repaglinide to about 0.6 gram per milliliter of the haloalkane solvent. In one particular variant, the concentration of the starting solution ranges is about 0.5 gram of S-repaglinide per milliliter of the haloalkane solvent. The ratio of the haloalkane to the C₅-C₁₀ aliphatic or alicyclic hydrocarbon, measured volume-to-volume, preferably ranges from about 1:1 to about 1:5. In one particular variant, the ratio of the haloalkane to the C₅-C₁₀ aliphatic or alicyclic hydrocarbon, measured volume-to-volume, is about 1:3. The formation of the starting solution of S-repaglinide in the haloalkane may be accomplished in a number of ways. For example, the step of forming the solution may involve mixing a powder of S-repaglinide with the haloalkane solvent. The powder of starting repaglinide may be in any form, including its crystalline Forms I and II, and amorphous S-repaglinide. Of course, any other forms of repaglinide may be used, including crude material, whether in solid or oil form. The isolated precipitate may be dried to obtain a powder of the crystalline Form III of S-repaglinide.

In a particular embodiment, the process for preparation of the crystalline Form III of S-repaglinide includes dissolving S-repaglinide in dichloromethane; adding petroleum ether to the solution to form a precipitate; and isolating the precipitate. In this embodiment, the preferred concentration of the dichloromethane solution ranges from about 0.4 to about 0.6 gram of S-repaglinide per milliliter of dichloromethane, and the preferred ratio of dichloromethane to petroleum ether, measured volume-to-volume, ranges from about 1:1 to about 1:5.

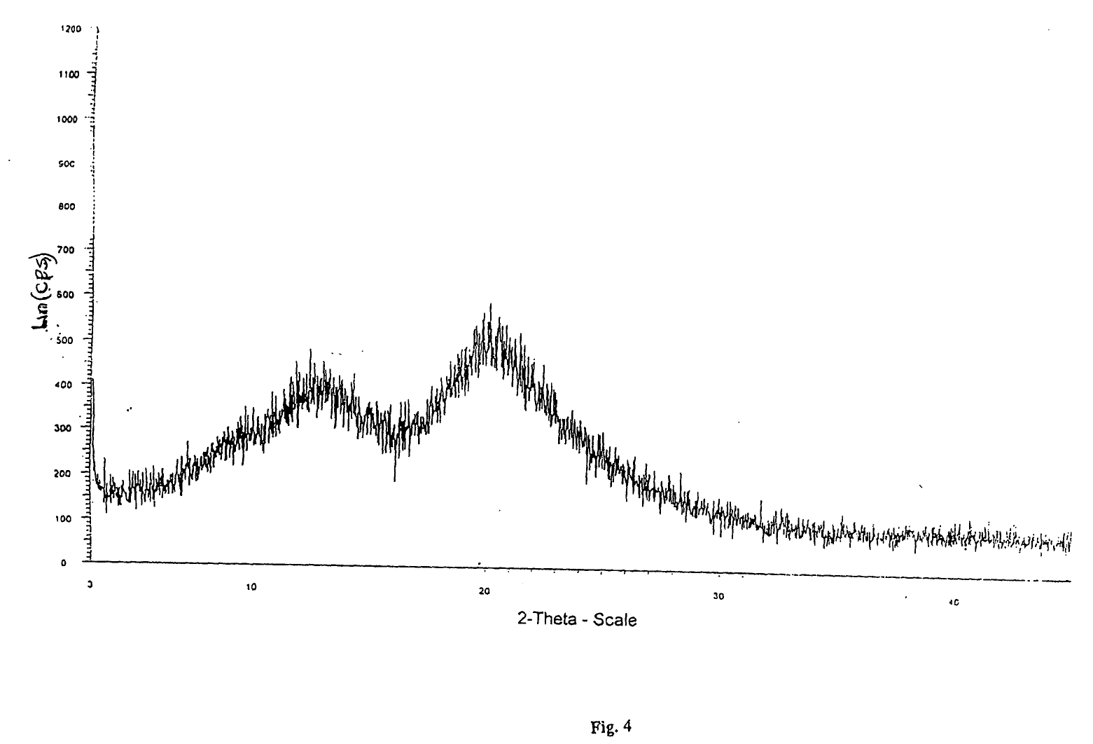

In another aspect, the invention also provides an amorphous form of (S)-repaglinide. While the invention is not limited to any specific theory, the inventors found that re-precipitation of S-repaglinide from a C₁-C₄, straight or branched chain, alcohol solvent provides solid S-repaglinide that is amorphous. FIG. 4 shows the X-ray powder diffractogram of the amorphous form of (S)-Repaglinide measured on a Bruker Axs, D8 Advance Powder X-ray Diffractometer with Cu K alpha-I Radiation source. As evident from FIG. 4, the powder obtained by the inventors is amorphous. The preferred alcohol solvents are methanol, ethanol, n-propanol, iso-propanol, n-butanol, iso-butanol and tertiary butanol, more preferably, methanol. In another aspect, a process for making the amorphous form of S-repaglinide is provided and includes forming a solution of S-repaglinide in a lower alcohol; cooling said solution so that a solid mass separates; and isolating the separated solid mass, which is the amorphous form of S-repaglinide. Preferably, the isolated solid mass is then dried to obtain a dry powder of amorphous S-repaglinide. Preferably, the step of forming the solution involves mixing a powder of the starting S-repaglinide and the lower alcohol, and heating the mixture to a temperature of from about 35° C. to about 70° C., preferably, to from about 45° C. to about 55° C., until the solution is formed. The solution may then be filtered to remove extraneous matter. The formation of the starting solution of S-repaglinide in the alcohol solvent may be accomplished in a number of ways. The powder of the starting S-repaglinide may be in any form, including its crystalline Forms I, II, and III. Of course, any other forms of repaglinide may be used, including crude material, whether in solid or oil form. The amorphous form of S-repaglinide produced by the processes described herein is also contemplated.

In a particular variant, the process for preparation of the amorphous form of (S)-repaglinide includes dissolving crystalline form of (S)-Repaglinide in the C₁-C₄ straight or branched chain alcoholic solvents at a temperature of 35-70° C., preferably at 45-55° C.; cooling the solution to a temperature of 0-5° C.; stirring the solution until a solid mass substantially separates therefrom; and filtering the separated solid; and drying the solid under vacuum at a temperature of 40 to 70° C. to a constant weight. The novel amorphous form of (S)-repaglinide obtained as per the above process is found to be free flowing and non-solvated and thus well suited for pharmaceutical applications.

Also provided are pharmaceutical compositions containing a crystalline Form III of repaglinide and a pharmaceutical carrier. In addition to the active compound, the pharmaceutical composition includes one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk, but ultimately in the form of a unit-dose formulation (i.e., a physically discrete unit containing a specific amount of active ingredient) such as a tablet or capsule. The pharmaceutical compositions may include, in addition to a compound of this invention, one or more active pharmaceutical compounds.

Generally, the pharmaceutical compositions are prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

The more preferred oral solid preparation is a tablet. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent, are suitable in the case of oral solid dosage forms (e.g., powders, capsules, and tablets). If desired, tablets may be coated by standard techniques. The compounds of this invention may be formulated into typical disintegrating tablet, or into a controlled or extended release dosage forms. Examples of suitable controlled release formulation vehicles are disclosed in U.S. Pat. Nos. 3,845,770; 3,916,899; 3,536,809; 3,598,123; and 4,008,719, incorporated herein by reference in their entireties.

The pharmaceutical compositions are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form, and may be prepared by methods well known in the art for a given formulation.

The amount of active ingredient included in a unit dosage form depends on the type of formulation in which the active ingredient is presented. A pharmaceutical composition will generally contain about 0.1% by weight to about 99% by weight of active ingredient, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a pre-determined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. The amount of active ingredient per unit dosage of solid formulations may be as described in prior art for preparations of S-repaglinide. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and poyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations of the present invention suitable for parenteral administration comprise sterile aqueous and non-aqueous injection solutions of the active compound, preferably isotonic with the blood of the intended recipient. The amount of active ingredient is preferably a concentration of from about 0.1% by weight to 10% by weight.

These preparations may contain, among other ingredients, anti-oxidants, buffers, bacteriostats, and solutes that render the formulation isotonic with the blood of the intended recipient. Aqueous and non-aqueous sterile suspensions may include, among others, suspending and thickening agents. The formulations may be presented in unit-dose or multi-dose containers, e.g., sealed capsules and vials, and may be stored in a freeze-dried or lyophilized condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by admixing the active compound with one or more conventional solid carriers, e.g., cocoa butter, and then shaping the resulting mixture.

Formulations suitable for transdermal delivery include ointments, creams, lotions, and oils and contain well-known pharmaceutically and cosmetically suitable ingredients. Bases for such formulations include for example alcohols, lanolin, petrolatum, paraffin, polyethylene glycol, emulsifiers, penetration enhancing agents, and oleaginous vehicles such as oils. Skin patches may also be used, typically consisting of a fabric or paper base impregnated with a suitable dose in a transdermal formulation. Formulations suitable for transdermal administration may also be delivered by iontophoresis, and typically take the form of an optionally buffered aqueous solution of the active compound.

The compounds of this invention may be combined with or linked to other compounds to obtain desired properties, for example the compounds of this invention may be linked to a stabilizing polymer such as a polyalkylene glycol (such as polyethylene glycol), or linked to a targeting compound such as an antibody. The resulting linked compounds are also part of this invention.

In another aspect, the invention also provides methods of treatment using the compounds and the pharmaceutical compositions of this invention. The compounds and compositions of this invention may be administered to a subject in an amount effective to stimulate insulin release by said subject. Further, the compounds and compositions of this invention may be administered to a subject for treating a disorder related to insulin release by administering to a subject an amount effective to stimulate insulin release by said subject. Methods for treating diabetes in a subject by administering a compound or composition of this invention to a subject in an amount effective to eliminate or alleviate symptoms of diabetes, or to prevent excessive blood sugar levels or reduce blood sugar levels, are also part of this invention. Methods for regulating blood sugar levels in a subject by administering an amount of a compound or composition of this invention effective to regulate blood sugar levels in the subject are also part of this invention.

In general, the treatment may be determined to alleviate, to eliminate, or to prevent a given condition based on factors determinable by a skilled physician as discussed below in the context of determining an effective amount for dosage.

By subject is meant a human or an animal, preferably human. Animals contemplated by this invention include any animal safely treatable by compounds of this invention, preferably mammals such as bovines, ovines, caprines, equines, felines, canines, rodents, leporids, and other mammalian farm and zoo animals or domestic pets.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. Particular formulation of active compound administered, the daily dose may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. An example of a dose per unit is 0.5 to 1 g. Another example of a dose per unit is 0.1 to 2 g.

The invention is further defined by reference to the following examples describing in detail the preparation of the compound and the compositions of the present invention, as well as their utility. It will be apparent to those skilled in the art, that many modifications, both to materials, and methods, may be practiced without departing from the purpose and interest of this invention. The examples that follow are not intended to limit the scope of the invention as defined hereinabove or as claimed below.

### EXAMPLE 1

Crude (S)-Repaglinide (5.0 grams) was dissolved in dichloromethane (10 ml) at an ambient temperature and stirred until a clear solution was obtained. The reaction mixture was stirred to get a clear solution. Petroleum ether (30 ml) was added to the reaction mixture and the reaction solution was cooled to a temperature of 0-5° C. The reaction mixture was stirred for about 1 hour to crystallize the solid mass. The solid mass was filtered, washed with petroleum ether (10.0 ml) and dried at a temperature of 50-55° C to afford the crystalline Form-III of S-Repaglinide. (Weight: 4.0 grams, Melting range: 80-84° C., M. C by KF: 0.35%).

### EXAMPLE 2

Crystalline Form-I of (S)-Repaglinide [recrystallised from acetone and petroleum ether] (10.0 grams) was dissolved in dichloromethane (20 ml) at an ambient temperature and stirred until a clear solution was obtained. Petroleum ether (60 ml) was added to the reaction mixture and the reaction solution was cooled to a temperature of 0-5° C. The reaction mixture was stirred for about 1 hour to crystallize the solid mass. The solid mass was filtered and washed with Petroleum ether (20.0 ml) and dried at a temperature of 50-55° C. to afford crystalline Form-III of S-Repaglinide. (Weight: 9.2 grams, Melting range: 80-84° C., M. C by KF: 0.25%).

### EXAMPLE 3

Crystalline Form-I of (S)-Repaglinide [recrystallised from aqueous ethanol] (10.0 grams) was dissolved in dichloromethane (20 ml) at an ambient temperature and stirred until a clear solution was obtained. Petroleum ether (60 ml) was added to the reaction solution and the solution was cooled to a temperature of 0-5° C. The reaction mixture was stirred for about 1 hour to crystallize the solid mass. The solid mass was filtered and washed with Petroleum ether (20.0 ml) and dried at a temperature of 50-55° C. to afford the crystalline Form-III of S-Repaglinide. (Weight: 7.8 grams, Melting range: 80-84° C., M. C by KF: 0.26%).

### EXAMPLE 4

Crystalline Form-II of (S)-Repaglinide [recrystallised from toluene and petroleum ether] (5.0 grams) was dissolved in dichloromethane (10 ml) at an ambient temperature and stirred to get a clear solution. Petroleum ether (30 ml) was added to the reaction mixture and the solution was cooled to a temperature of 0-5° C. The reaction mixture was stirred for about 1 hour to crystallize the solid mass. The solid mass was filtered and washed with Petroleum ether (10.0 ml) and dried at a temperature of 50-55° C. to afford the crystalline Form-Ill of S-Repaglinide. (Weight: 4.8 grams, Melting range: 80-83° C., M. C by KF: 0.2%).

### EXAMPLE 5

Crystalline Form-II of (S)-Repaglinide [recrystallised from toluene] (20.0 grams) was dissolved in dichloromethane (40 ml) at an ambient temperature and stirred till a clear solution was obtained. Petroleum ether (120 ml) was added to the reaction mixture and the solution was cooled to a temperature of 0-5° C. The reaction mixture was stirred for about 1 hour to crystallize the solid mass. The solid mass was filtered and washed with Petroleum ether (40.0 ml) and dried at a temperature of 50-55° C. to afford the crystalline Form-III of S-Repaglinide. (Weight: 19.2 grams, Melting range: 80-84° C., M. C by KF: 0.3%)

### EXAMPLE 6

Amorphous form of (S)-Repaglinide (10.0 grams) was dissolved in dichloromethane (20 ml) at an ambient temperature and stirred until a clear solution was obtained. Petroleum ether (60 ml) was added to the reaction solution and then cooled to a temperature of 0-5° C. The reaction mixture was stirred for about 1 hour to crystallize the solid mass. The solid mass was filtered and washed with Petroleum ether (20.0 ml) and dried at a temperature of 50-55° C. to afford the crystalline Form-III of S-Repaglinide. (Weight: 8.6 grams, Melting range: 79-84° C., M. C by KF: 0.20%).

### EXAMPLE 7

Crystalline Form-I [recrystallised from acetone and petroleum ether] of (S)-Repaglinide (6.0 grams) was dissolved in methanol (20.0 ml) and heated to a temperature of 45-50° C. till the clear solution was obtained; further the solution was cooled to a temperature of 0-5° C. and stirred to crystallize the solid mass. The solid mass was filtered, washed with methanol (12.0 ml) and dried at a temperature of 50-60° C. under vacuum to afford the amorphous form of (S)-Repaglinide. (Weight: 5.4 grams, Melting range: 74-77° C., M. C by KF: 0.82%).

### EXAMPLE 8

Crystalline Form-II [recrystallised from toluene and petroleum ether] of (S)-Repaglinide (10.0 grams) was dissolved in methanol (34.0 ml) and heated to a temperature of 45-50° C. till the clear solution was obtained; further the solution was cooled to a temperature of 0-5° C. and stirred to crystallize the solid mass. The solid mass was filtered, washed with methanol (20-0 ml) and dried at a temperature of 50-60° C. under vacuum to afford the novel amorphous form of (S)-Repaglinide. (Weight: 9.4 grams, Melting range: 74-76° C., M. C by KF: 0.01%).

### EXAMPLE 9

Crystalline Form-II [recrystallised from toluene] of (S)-Repaglinide (20.0 grams) was dissolved in methanol (67.0 ml) and heated to a temperature of 45-50° C. till the clear solution was obtained; further the reaction solution was cooled to a temperature of 0-5° C. and stirred to crystallize the solid mass. The separated solid was filtered, washed with methanol (40.0 ml) and dried at a temperature of 50-60° C. under vacuum to afford the amorphous form of (S)-Repaglinide. (Weight: 18.2 grams, Melting range: 73-76° C., M. C by KF: 0.90%).

### EXAMPLE 10

Crystalline Form-III of (S)-Repaglinide (20.0 grams) was dissolved in methanol (67.0 ml) and heated to a temperature of 45-50° C. till the clear solution was obtained; further the solution was cooled to a temperature of 0-5 C and stirred to crystallize the solid mass. The solid mass was filtered, washed with methanol (40.0 ml) and dried at a temperature of 50-60° C. under vacuum to afford the amorphous form of (S)-Repaglinide. (Weight: 17.2 grams, Melting range: 74-77° C., M. C by KF: 0.92%).

### EXAMPLE 11

Crude (S)-Repaglinide (100.0 grams) was dissolved in toluene (2.0 liters) and heated to a temperature of 70-75° C. till the clear solution was obtained; further the reaction solution was cooled to a temperature of 10-15° C. and stirred to crystallize the solid mass. Then solid mass was filtered, washed with toluene (200.0 ml) and dried at a temperature of 50-60° C. under vacuum to afford the crystalline Form-II of (S)-Repaglinide. (Weight: 77.4 grams, Visual Melting Point: 101-104° C., M. C. by KF: 0.18%).

### EXAMPLE 12

Crystalline Form-I of (S)-Repaglinide (5.0 grams) was dissolved in toluene (100.0 ml) and heated to a temperature of 60-65° C. till the clear solution was obtained. Then the reaction solution was cooled to a temperature of 10-15° C. and stirred to crystallize the solid mass. The solid mass was filtered, washed with toluene (10.0 ml) and dried at a temperature of 50-60° C. under vacuum to afford the crystalline Form-II of (S)-Repaglinide. (Weight: 4.4 grams, M. C. by KF: 0.16%).

### EXAMPLE 13

Crystalline Form-III of (S)-Repaglinide (20.0 grams) was dissolved in toluene (400.0 ml) and heated to a temperature of 60-65° C. till the clear solution was obtained. Then the reaction solution was cooled to a temperature of 10-15° C. and stirred to crystallize the solid mass. The solid mass was filtered, washed with toluene (40.0 ml) and dried at a temperature of 50-60° C. under vacuum to afford the crystalline Form-II of (S)-Repaglinide. (Weight: 18.2 grams, M. C. by KF: 0.15%).

### EXAMPLE 14

Amorphous form of (S)-Repaglinide (10.0 grams) was dissolved in toluene (200.0 ml) and heated to a temperature of 60-65° C. till the clear solution was obtained. Then the reaction solution was cooled to a temperature of 10-15° C. and stirred to crystallize the solid mass. The solid mass was filtered, washed with toluene (10.0 ml) and dried at a temperature of 50-60° C. under vacuum to afford the crystalline Form-II of (S)-Repaglinide. (Weight: 9.4 grams, M. C. by KF: 0.06%).

### EXAMPLE 15

Repaglinide (5.0 grams) was dissolved in acetone (25 ml) at a temperature of 30° C. and stirred till the clear solution was obtained. Petroleum ether (25.0 ml+′5.0 ml) was added to the reaction solution and the reaction solution was and stirred for about 70 minutes at a temperature of 35° C. to crystallize the solid mass. The solid mass was filtered, washed with petroleum ether (10.0 ml) under reduced pressure, and dried at a temperature of 50-70° C. to afford 4.0 grams of crystalline Form-I of Repaglinide.

### EXAMPLE 16

Crude Repaglinide (20.0 grams) was dissolved in methanol (80.0 ml) at a temperature of 30° C. and stirred till a clear solution was obtained. Water (80.0 ml) was added to the reaction solution and stirred for about 4 hours at a temperature of 30° C. to crystallize the solid mass. The solid mass was filtered, washed with water (40.0 ml) under reduced pressure, and dried at a temperature of 40-50° C. to a constant weight to afford 17.2 grams of crystalline Form-I of Repaglinide.

### EXAMPLE 17

Repaglinide (5.0 grams) was dissolved in tetrahydrofuran (20 ml) at a temperature of 30° C. and stirred till the clear solution was obtained. Methyl-ter-butyl ether (30.0 ml÷30.0 ml) was added to the reaction solution and the solution was cooled to a temperature of 0-5° C. and stirred for about 1.5 hours to crystallize the solid mass. The solid mass was filtered, washed with cold methyl-ter-butyl ether (10.0 ml) under reduced pressure and dried at a temperature of 40-50° C. to a constant weight to afford the crystalline Form-I of Repaglinide.

### EXAMPLE 18

Repaglinide (5.0 grams) was dissolved in ethyl acetate (20.0 ml+30.0 ml) in a portion wise and stirred to get a clear solution at a temperature of 40-50° C. Petroleum ether (50 ml) was added to the reaction solution and cooled to a temperature of 0-5° C. and stirred for about 1.5 hours to crystallize the solid mass. The solid mass was filtered, washed with cold petroleum ether (10.0 ml) under reduced pressure and dried at a temperature of 40-50° C. to a constant weight to afford the crystalline Form-I of Repaglinide.

### EXAMPLE-19

Repaglinide (5.0 grams) was dissolved in n-propanol (25.0 ml) at a temperature of 40-50° C. and stirred till a clear solution was obtained. Water (50 ml) was added to the reaction solution and the resultant reaction mixture was cooled to a temperature of 0-5° C. and stirred for about 1.5 hours to crystallize the solid mass. The solid mass was filtered, washed with cold water (10.0 ml) under reduced pressure and dried at a temperature of 40-50° C. to a constant weight to afford 3.6 grams of crystalline Form-I of Repaglinide.

### EXAMPLE 20

Repaglinide (5.0 grams) was dissolved in acetonitrile (25.0 ml) at a temperature of 40-50° C. and stirred till a clear solution was obtained. Water (50 ml) was added to the reaction solution and the resultant reaction mixture was cooled to a temperature of 0-5° C. and the solution was stirred for a period of about 1.5 hours to crystallize the solid mass. The solid mass was filtered, washed with cold water (10.0 ml) under reduced pressure and dried at a temperature of 40-50° C. to a constant weight to afford 3.6 grams of crystalline Form-I of Repaglinide.

### EXAMPLE 21

Repaglinide (20.0 grams) was dissolved in methyl isobutyl ketone (30.0 ml+10.0 ml) at a temperature of 40-50° C. in a portion wise manner and stirred for about 1.5 hours till a clear solution was obtained. Methyl iter-butyl ether (80.0 ml) was added to above clear solution, the resultant solution was cooled to a temperature of 0-5° C. accompanied by stirring to crystallize the solid mass. The solid mass was filtered,—washed with methyl ter-butyl ether (10.0 ml) under reduced pressure and dried at a temperature of 40-50° C. to a constant weight to afford 3.4 grams of crystalline Form-I of Repaglinide.

### EXAMPLE 22

Repaglinide (5.0 grams) was dissolved in diethyl ketone (30.0 ml) at a temperature of 40-50° C. and stirred till a clear solution was obtained. Methyl ter-butyl ether (40.0 ml) was added to the reaction solution and the resultant reaction mixture was cooled to a temperature of 0-5° C. and stirred for about 1.5 hours to crystallize the solid mass. The solid mass was filtered, washed with methyl ter-brityl ether (10.0 ml) under reduced pressure and dried at a temperature of 40-50° C. to a constant weight to afford 4.2 grams of crystalline Form-I of Repaglinide.

### EXAMPLE 23

Repaglinide (5.0 grams) was dissolved in ter-butanol (30.0 ml) at a temperature of 40-50° C. and stirred till a clear solution was obtained. Water (50.0 ml) was added to the reaction solution and the resultant reaction mixture was cooled to a temperature of 0-5° C. and stirred for about 1.5 hours to crystallize the solid mass. The solid mass was filtered; washed with water (10.0 ml) under reduced pressure and dried at a temperature of 40-50° C. to a constant weight to afford 4.6 grams of crystalline Form-I of Repaglinide.

### EXAMPLE 24

Repaglinide (5.0 grams) was dissolved in methyl ethyl ketone (20.0 ml) at a temperature of 40-50° C. and stirred till a clear solution was obtained. N-heptane (30.0 ml) was slowly added to the reaction solution and the resultant reaction mixture was cooled to a temperature of 0-5° C. and stirred for about 1 hour to crystallize the solid mass. The solid mass was filtered, washed with cold n-heptane (10.0 ml) under reduced pressure and dried at a temperature of 40-50° C. to a constant weight to afford 3.6 grants of crystalline Form-I of Repaglinide.

### EXAMPLE 25

Repaglinide (5.0 grams) was dissolved in diglyme (20.0 ml) at a temperature of 40-50° C. and stirred till a clear solution was obtained. N-heptane (30.0 ml)) was slowly added to above clear solution and stirred for about 1 hour at 40° C., then the resultant reaction mixture was cooled to a temperature of 0-5° C. and stirred for 30 minutes to crystallize the solid mass. The solid mass was filtered, washed with cold n-heptane (10.0 ml) under reduced pressure and dried at a temperature of 40-50° C. to a constant weight to afford 3.2 grams of crystalline Form-I of Repaglinide.

### EXAMPLE 26

Repaglinide (5.0 grams) was dissolved in methyl ethyl ketone (20.0 ml) at a temperature of 40-50° C. and stirred till a clear solution was obtained. Methyl ter-butyl ether (30.0 ml) was added to the reaction solution and stirred for 30 minutes at a temperature of 40° C., then the resultant reaction mixture was cooled to a temperature of 0-5° C. and stirred for about 1.5 hours to crystallize the solid mass. The solid mass was filtered, washed with cold methyl ter-butyl ether (10.0 ml) under reduced pressure and dried at a temperature of 40-50° C. to a constant weight to afford 3.2 grams of crystalline Form-I of Repaglinide.

### EXAMPLE 27

1001041 Repaglinide (5.0 grams) was dissolved in 1,4 dioxane (20.0 ml) at a temperature of 40-50° C. and stirred till a clear solution was obtained. N-heptane (30.0 ml) was slowly added to the reaction solution and the solution was cooled to a temperature of 0-5° C. and stirred for about 1 hour to crystallize the solid mass. The solid mass was filtered, washed with n-heptane (10.0 ml) under reduced pressure and dried at a temperature of 40-50° C. to a constant weight to afford 3.6 grams of crystalline Form-1 of Repaglinide.

### EXAMPLE 28

Repaglinide (5.0 grams) was dissolved in n-butanol (20.0 ml) at a temperature of 40-50° C. and stirred to get a clear solution. Methyl ter-butyl ether (40.0 ml) was added to the reaction solution and the solution was cooled to a temperature of 0-5° C. and stirred for about 1 hour to crystallize the solid mass. The solid mass was filtered, washed with methyl ter-butyl ether (10.0 ml) under reduced pressure aid dried at a temperature of 40-50° C. to a constant weight to afford 4.0 grams of crystalline Form-I of Repaglinide.

### EXAMPLE 29

Crude Repaglinide (20.0 grams) was dissolved in iso-butanol (20.0 ml) at a temperature of 40-50° C. and stirred till a clear solution was obtained. N-hexane (100.0 ml) was added to the reaction mixture and the solution was cooled to a temperature of 0-5° C. and stirred for about 1.5 hours to crystallize the solid mass. The solid mass was filtered, washed with n-hexane (10.0 ml) under reduced pressure and dried at a temperature of 40-50° C. to a constant weight to afford 17.4 grams of crystalline Form-I of Repaglinide.

Unless stated to the contrary, words and phrases such as “including,” “containing,” “comprising,” “having”, “for example”, “i.e.”, “in particular” and the like, means “including without limitation” and shall not be construed to limit any general statement that it follows to the specific or similar items or matters immediately following it. Except where the context indicates to the contrary, all exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. A compound which is a crystalline Form III of S-repaglinide.

2. The compound of claim 1, having an X-ray diffraction pattern, expressed in terms of 2 theta angles, that includes five or more peaks selected from the group consisting of 4.44±0.09, 6.81±0.09, 7.80±0.09, 9.28±0.09, 11.09±0.09, 11.89±0.09, 12.92±0.09, 13.46±0.09, 14.34±0.09, 15.77±0.09, 16.24±0.09, 17.08±0.09, 18.06±0.09, 18.75±0.09, 19.25±0.09, 19.59±0.09, 19.99±0.09, 20.34±0.09, 21.18±0.09, 21.96±0.09, 22.18±0.09, 22.58±0.09, 23.24±0.09, 23.77±0.09, 24.08±0.09, 25.02±0.09, 25.31±0.09, 25.78±0.09, 26.67±0.09, 27.39±0.09, 28.03±0.09, 30.26±0.09, 35.50±0.09, and 38.74±0.09 degrees.

3. The compound of claim 1, having substantially the same X-ray diffraction pattern as shown in FIG. 1.

4. The compound of claim 1, having a differential scanning calorimetry thermogram which exhibits a significant endotherm peak at about 80° C.

5. The compound of claim 4, having substantially the same differential scanning calorimetry thermogram as shown in FIG. 2.

6. The compound of claim 1, having an infrared absorption spectrum with absorption bands at about 3291 cm⁻¹, about 3029 cm⁻¹, about 2935 cm⁻¹, about 2795 cm⁻¹, about 1292 cm⁻¹, about 1727 cm⁻¹, about 1643 cm⁻¹, about 1611 cm¹, about 1537 cm¹, about 1436 cm⁻¹, about 1225 cm⁻¹, about 1171 cm⁻¹, about 1087 cm⁻¹, about 1028 cm⁻¹, about 986 cm⁻¹, about 922 cm⁻¹, about 860 cm⁻¹, about 764 cm⁻¹, about 686 cm⁻¹, and about 533 cm⁻¹.

7. The compound of claim 6, having substantially the same infrared spectrum as that shown in FIG. 3.

8. A composition comprising S-repaglinide as a solid, wherein at least 80% by weight of said solid S-repaglinide is its crystalline Form III having an X-ray diffraction pattern, expressed in terms of 2 theta angles, that includes five or more peaks selected from the group consisting of 4.44±0.09, 6.81±0.09, 7.80±0.09, 9.28±0.09, 11.09±0.09, 11.89±0.09, 12.92±0.09, 13.46±0.09, 14.34±0.09, 15.77±0.09, 16.24±0.09, 17.08±0.09, 18.06±0.09, 18.75±0.09, 19.25±0.09, 19.59±0.09, 19.99±0.09, 20.34±0.09, 21.18±0.09, 21.96±0.09, 22.18±0.09, 22.58±0.09, 23.24±0.09, 23.77±0.09, 24.08±0.09, 25.02±0.09, 25.31±0.09, 25.78±0.09, 26.67±0.09, 27.39±0.09, 28.03±0.09, 30.26±0.09, 35.50±0.09, and 38.74±0.09 degrees.

9. The composition of claim 8, wherein at least 90% by weight of said solid S-repaglinide is the crystalline Form III.

10. The composition of claim 8, wherein at least 95% by weight of said solid S-repaglinide is the crystalline Form III.

11. The composition of claim 8, wherein at least 99% by weight of said solid S-repaglinide is the crystalline Form III.

12. The composition of claim 8, wherein said solid S-repaglinide is substantially free of its crystalline Forms I and II.

13. The composition of claim 8, wherein at least 1% of said solid S-repaglinide is not the crystalline Form III.

14. The composition of claim 8, wherein at least 5% of said solid S-repaglinide is not the crystalline Form III.

15. A pharmaceutical composition comprising a) the compound of claim 1, and b) a pharmaceutically acceptable carrier or diluent.

16. The pharmaceutical composition of claim 15, further comprising one or more pharmaceutically acceptable excipients.

17. The pharmaceutical composition of claim 16, which is a solid dosage form for oral administration.

18. The pharmaceutical composition of claim 17, wherein said solid dosage form is a tablet.

19. A process for preparation of a crystalline Form III of S-repaglinide, said process comprising:
a. providing a solution of S-repaglinide in a haloalkane solvent;
b. contacting said solution with C₅-C₁₀ aliphatic or alicyclic hydrocarbon anti-solvent thereby forming a precipitate; and
c. isolating the precipitate, which is the crystalline Form III of S-repaglinide.

20. The process of claim 19, further comprising drying the isolated precipitate.

21. The process of claim 19, wherein the providing step includes mixing a powder of the starting S-repaglinide with the haloalkane solvent to form said solution.

22. The process of claim 21, wherein said powder of the starting S-repaglinide is a solid form of S-repaglinide selected from the group consisting of crystalline Form I, crystalline Form II and amorphous S-repaglinide.

23. The process of claim 19, wherein the haloalkane solvent is selected from the group consisting of dichloromethane, chloroform, and dichloroethane.

24. The process of claim 19, wherein the C₅-C₁₀ aliphatic or alicyclic hydrocarbon is a C₅-C₇ aliphatic or alicyclic hydrocarbon.

25. The process of claim 19, wherein the C₅-C₁₀ aliphatic or alicyclic hydrocarbon is selected from the group consisting of petroleum ether, hexane, n-heptane, cyclohexane, and cycloheptane.

26. The process of claim 19, wherein the concentration of said solution is from about 0.25 gram to about 1 gram per milliliter of the haloalkane solvent.

27. The process of claim 26, wherein the concentration of said solution is from about 0.4 gram to about 0.6 gram of S-repaglinide per milliliter of the haloalkane.

28. The process of claim 27, wherein the concentration of said solution is about 0.5 gram of S-repaglinide per milliliter of the haloalkane.

29. The process of claim 19, wherein the ratio of said haloalkane to said C₅-C₁₀ aliphatic or alicyclic hydrocarbon, measured volume-to-volume, ranges from about 1:1 to about 1:5.

30. The process of claim 19, wherein said ratio of said haloalkane to said C₅-C₁₀ aliphatic or alicyclic hydrocarbon is about 1:3.

31. The process of claim 19, wherein the contacting step includes adding said C₅-C₁₀ aliphatic or alicyclic hydrocarbon to said solution.

32. The process of claim 19, wherein said C₅-C₁₀ aliphatic or alicyclic hydrocarbon is petroleum ether.

33. The process of claim 32, wherein said haloalkane is dichloromethane.

34. A compound which is the crystalline Form III of S-repaglinide produced by the process of claim 19.

35. A compound which is the crystalline Form III of S-repaglinide produced by the process of claim 33.

36. A process for preparation of a crystalline Form III of S-repaglinide, said process comprising:
a) dissolving S-repaglinide in dichloromethane;
b) adding petroleum ether to the solution to form a precipitate; and
c) isolating the precipitate, which is the crystalline Form III of S-repaglinide.

37. The process of claim 36, wherein the concentration of the dichloromethane solution is from about 0.4 to about 0.6 gram of S-repaglinide per milliliter of dichloromethane, and the ratio of dichloromethane to petroleum ether, measured volume-to-volume, ranges from about 1:1 to about 1:5.

38. A compound which an amorphous form of S-repaglinide.

39. The compound of claim 1 having substantially the same X-ray diffraction pattern as shown in FIG. 4.

40. A process for making an amorphous form of S-repaglinide, said process comprising:
a) providing S-repaglinide as a solution in a lower alcohol;
b) cooling said solution so that a solid mass separates;
c) isolating said separated solid mass, which is the amorphous form of S-repaglinide.

41. The process of claim 40, further comprising drying said isolated solid mass.

42. The process of claim 40, wherein said providing step includes mixing a powder of the starting S-repaglinide and the lower alcohol, and heating the mixture to a temperature of from about 35° C. to about 70° C. until the solution is formed.

43. The process of claim 40, wherein the mixture is heated to from about 45° C. to about 55° C.

44. The process of claim 40, wherein the solution of S-repaglinide is cooled to from about 0° C. to about 5° C.

45. The process of claim 41, wherein said powder of the starting S-repaglinide is selected from the group consisting of crystalline Form I, crystalline Form II and crystalline Form III.

46. The process of claim 40, wherein the lower alcohol is selected from the group consisting of methanol, ethanol, n-propanol, isopropanol, n-butanol, isobutanol, and t-butanol.

47. The process of claim 40, wherein the lower alcohol is methanol.

48. A compound which the amorphous form of repaglinide produced by a process of claim 40.

49. A compound which the amorphous form of repaglinide produced by a process of claim 48.

50. A process for preparation of a crystalline Form II of S-repaglinide, said process comprising:
a) providing a solution of S-repaglinide in a solvent containing aromatic hydrocarbon with the proviso that said solvent does not include petroleum ether;
b) cooling said solution thereby a solid mass separates;
c) isolating said solid mass, which is said crystalline Form II of S-repaglinide.

51. The process of claim 50, wherein said solvent does not include any aliphatic hydrocarbon components.

52. The process of claim 50, wherein said solvent consists of said aromatic hydrocarbon.

53. The process of claim 50, wherein said aromatic hydrocarbon is selected from the group consisting of benzene, toluene, ethyl benzene and xylene.

54. The process of claim 50, wherein said aromatic hydrocarbon is toluene.

55. The process of claim 52, wherein said aromatic hydrocarbon is toluene.

56. The process of claim 50, wherein the providing step includes mixing a powder of the starting S-repaglinide with the solvent and heating said mixture to form the solution.

57. The process of claim 50, further comprising drying the isolated solid mass.
